(19) United States
(12) Patent Application Publication
(10) Pub. No.: US 2026/0102060 A1
(43) Pub. Date: Apr. 16, 2026

(54) LIGHT SOURCE DEVICE AND ENDOSCOPE SYSTEM

(71) Applicant: SONY GROUP CORPORATION, TOKYO (JP)

(72) Inventors: TETSUAKI IWANE, TOKYO (JP); TOMOYUKI OKI, TOKYO (JP); SATOSHI NAGAE, TOKYO (JP)

(21) Appl. No.: 19/113,829

(22) PCT Filed: Sep. 22, 2023
(86) PCT No.: PCT/JP2023/034588

§ 371 Date: Mar. 20, 2025

(30) Foreign Application Priority Data
Sep. 29, 2022 (JP) 2022-157137

(51) Int. Cl. A61B 1/06; A61B 1/00; A61B 1/07; F21V 8/00; H04N 23/50

(52) U.S. Cl. CPC A61B 1/0646; A61B 1/00045; A61B 1/0684; A61B 1/07; G02B 6/0006; G02B 6/0008; H04N 23/555

(57) ABSTRACT

A light source device according to an embodiment includes an incident lens on which first light emitted from a first light source is made incident, a first light guide on which the first light emitted from the incident lens is made incident, a multiplexing unit that multiplexes the first light emitted from the first light guide and second light emitted from a second light source and makes multiplexed light incident on a second light guide, and a conversion element that diffuses incident light at a predetermined diffusion angle. The conversion element is provided between the incident lens and the first light guide.

### FIELD

The present disclosure relates to a light source device and an endoscope system.

### BACKGROUND

As an apparatus for viewing an internal structure of an object, an endoscope has been widely used. In particular, in the medical field, endoscopes have rapidly spread according to the development of surgical techniques and are now indispensable in many medical fields. In recent years, an endoscope has an additional function of performing fluorescence observation using a drug and can be used as equipment that supports surgical techniques of doctors.

In the endoscope system explained above enabled to perform the fluorescence observation, a technology for simultaneously irradiating white light and excitation light for performing the fluorescence observation and superimposing an affected part image obtained by the white light and a lesion image obtained by the fluorescence observation has been proposed. According to this technology, by displaying a lesion in real time and at a more accurate position, it is possible to realize advanced surgical support for doctors.

Patent Literature 1 discloses a medical light source device including a light source that emits excitation light and a light source that emits white light and including a configuration in which rays emitted from these light sources are multiplexed by optical systems inside the light sources and irradiated to a target.

### CITATION LIST

### Patent Literature

Patent Literature 1: WO 2020/036112 A

### SUMMARY

### Technical Problem

In the configuration explained above in which the rays of the excitation light and the white light are multiplexed and emitted, it is assumed that a narrowband light source that emits the excitation light is a laser diode (LD) and a wideband light source that emits the white light is a light emitting diode (LED). In this case, as a result of designing an imaging size of LD light at a light guide incident end to be small using characteristics of the LD, an imaging sizes of the LD light and the LED light are sometimes different.

In this state, when light guides having various diameters are connected to the light sources, a ratio of amounts of light taken into light guides of the LD light and the LED light changes according to the diameters of the light guides. When the light amount ratio of the LD light and the LED light after the multiplexing changes according to the diameters of the light guides, a ratio of brightness of the LD light and the LED light at the time of fluorescence observation changes. This affects observation image quality.

An object of the present disclosure is to provide a light source device and an endoscope system capable of, when light by narrowband light source and light by a wideband light source are multiplexed and emitted via a light guide, suppressing dependency of a light amount ratio of the light by the narrowband light source and the light by the wideband light source on a light guide diameter.

### Solution to Problem

For solving the problem described above, a light source device according to one aspect of the present disclosure has an incident lens on which first light emitted from a first light source is made incident; a first light guide on which the first light emitted from the incident lens is made incident; a multiplexing unit that multiplexes the first light emitted from the first light guide and second light emitted from a second light source and makes multiplexed light incident on a second light guide; and a conversion element that diffuses incident light at a predetermined diffusion angle, wherein the conversion element is provided between the incident lens and the first light guide.

### BRIEF DESCRIPTION OF DRAWINGS

FIG. 1 is a schematic diagram illustrating an example of a schematic configuration of an endoscope system.

FIG. 2 is a block diagram illustrating an example of functional components of a camera and a CCU in the endoscope system.

FIG. 3 is a schematic diagram illustrating an example of a schematic configuration of a microscopic surgery system.

FIG. 4 is a schematic diagram illustrating a configuration of an example of a light source device capable of simultaneously irradiating excitation light and white light according to an existing technology.

FIG. 5 is a schematic diagram illustrating an example of a relation between a diameter of an external light guide and a ratio of light amounts of narrowband light and wideband light according to the existing technology.

FIG. 6 is a schematic diagram illustrating a configuration of an example of a light source device according to a first embodiment.

FIG. 7 is a schematic diagram illustrating an example of a relation between a diameter of an external light guide and a ratio of light amounts of narrowband light and wideband light according to the first embodiment.

FIG. 8 is a schematic diagram illustrating an example of a light source device corresponding to a plurality of light sources that respectively emit narrowband light according to the first embodiment.

FIG. 9 is a schematic diagram illustrating a configuration of an example of a light source device according to a second embodiment.

FIG. 10 is a schematic diagram illustrating an example of a reflection characteristic of a bandpass filter in the case in which the reflectance of light having a wavelength including an excitation wavelength of a light source is designed to be low.

FIG. 11 is a schematic diagram illustrating a configuration of an example of a light source device according to a second example of the second embodiment.

FIG. 12 is a schematic diagram illustrating a configuration of an example of a light source device according to a third embodiment.

### DESCRIPTION OF EMBODIMENTS

Embodiments of the present disclosure are explained in detail below with reference to the drawings. Note that, in the embodiments explained below, redundant explanation is omitted by denoting the same parts with the same reference numerals and signs.

The embodiments of the present disclosure are explained in detail below according to the following order.
1. Endoscope system applicable to the embodiments of the present disclosure
2. Existing technology
3. Overview of the embodiments of the present disclosure
4. First embodiment of the present disclosure
5. Second embodiment of the present disclosure
5-1. First example of the second embodiment
5-2. Second example of the second Embodiment
6. Third embodiment of the present disclosure

Embodiments according to the present disclosure are explained below. The embodiments according to the present disclosure relate to an endoscope system that supports an operator by observing a surgical site with an endoscope inserted into an abdominal cavity in surgery of an abdominal cavity or the like. The embodiments according to the present disclosure relate to an endoscope system and, in particular, relates to a light source device that irradiates a surgical site to be observed by an endoscope with illumination light.

### (1. Endoscope System Applicable to the Embodiments of the Present Disclosure)

First, in order to facilitate understanding, an endoscope system applicable to the embodiments of the present disclosure is explained.

### (Endoscope System)

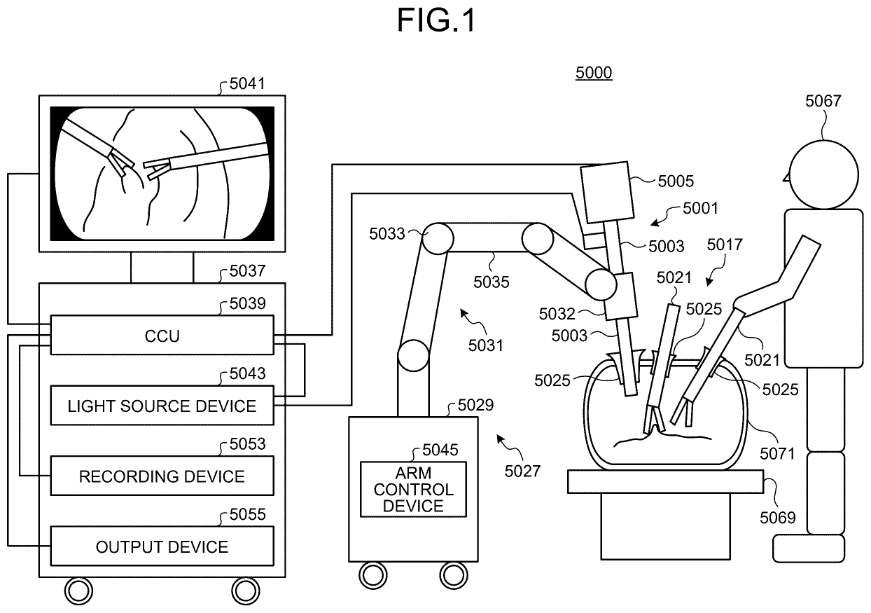

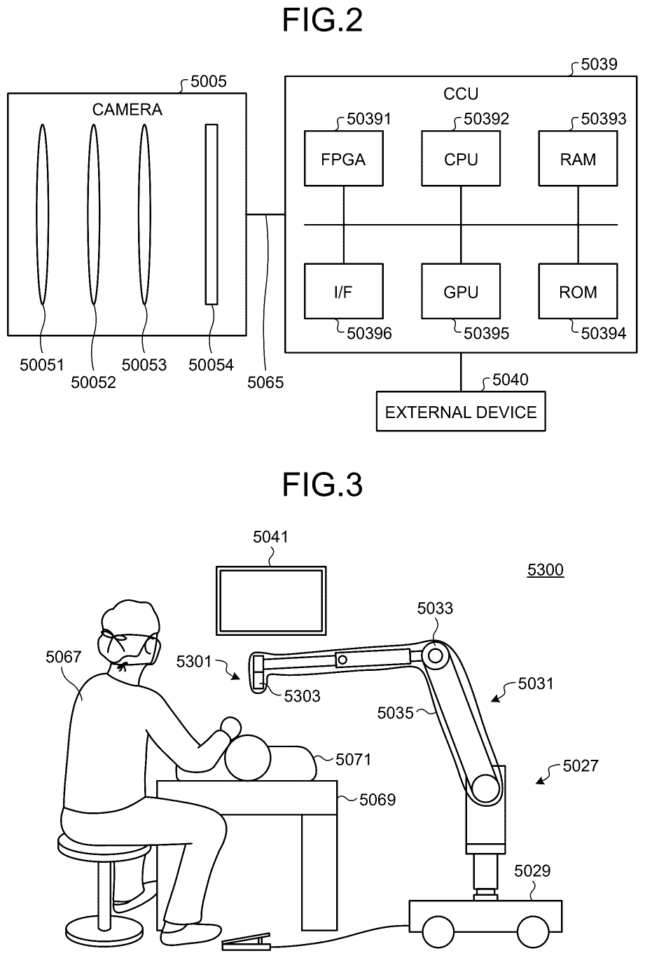

An example of the endoscope system will be described using FIGS. 1 and 2. FIG. 1 is a diagram illustrating an example of a schematic configuration of an endoscope system 5000 to which the technology according to the present disclosure is applicable. FIG. 2 is a diagram illustrating an example of a configuration of an endoscope 5001 and a camera control unit (CCU) 5039. FIG. 1 illustrates a situation where an operator (for example, a doctor) 5067 who is a participant of an operation performs the operation on a patient 5071 on a patient bed 5069 using the endoscope system 5000. As illustrated in FIG. 1, the endoscope system 5000 includes the endoscope 5001 that is a medical imaging device, the CCU 5039, a light source device 5043, a recording device 5053, an output device 5055, and a support device 5027 for supporting the endoscope 5001.

In endoscopic surgery, insertion assisting tools called trocars 5025 are punctured into the patient 5071. Then, a scope 5003 connected to the endoscope 5001 and surgical tools 5021 are inserted into a body of the patient 5071 through the trocars 5025. The surgical tools 5021 include: an energy device such as an electric scalpel; and forceps, for example.

A surgical image that is a medical image in which the inside of the body of the patient 5071 is captured by the endoscope 5001 is displayed on a display device 5041. The operator 5067 performs a procedure on a surgical target using the surgical tools 5021 while viewing the surgical image displayed on the display device 5041. The medical image is not limited to the surgical image, and may be a diagnostic image captured during diagnosis.

### (Endoscope)

The endoscope 5001 is an imaging section for capturing the inside of the body of the patient 5071, and is, for example, as illustrated in FIG. 2, a camera including a condensing optical system 50051 for condensing incident light, a zooming optical system 50052 capable of optical zooming by changing a focal length of the imaging section, a focusing optical system 50053 capable of focus adjustment by changing the focal length of the imaging section, and a light receiving sensor 50054. The endoscope 5001 condenses the light through the connected scope 5003 on the light receiving sensor 50054 to generate a pixel signal, and outputs the pixel signal through a transmission system to the CCU 5039. The scope 5003 is an insertion part that includes an objective lens at a distal end and guides the light from the connected light source device 5043 into the body of the patient 5071. The scope 5003 is, for example, a rigid scope for a rigid endoscope and a flexible scope for a flexible endoscope. The scope 5003 may be a direct viewing scope or an oblique viewing scope. The pixel signal only needs to be a signal based on a signal output from a pixel, and is, for example, a raw signal or an image signal. The transmission system connecting the endoscope 5001 to the CCU 5039 may include a memory, and the memory may store parameters related to the endoscope 5001 and the CCU 5039. The memory may be disposed at a connection portion of the transmission system or on a cable. For example, the memory of the transmission system may store the parameters before shipment of the endoscope 5001 or the parameters changed when current is applied, and an operation of the endoscope may be changed based on the parameters read from the memory. A set of the camera and the transmission system may be referred to as an endoscope. The light receiving sensor 50054 is a sensor for converting the received light into the pixel signal, and is, for example, a complementary metal-oxide-semiconductor (CMOS) imaging sensor. The light receiving sensor 50054 is preferably an imaging sensor having a Bayer array capable of color imaging. The light receiving sensor 50054 is also preferably an imaging sensor having a number of pixels corresponding to a resolution of, for example, 4K (3840 horizontal pixels×2160 vertical pixels), 8K (7680 horizontal pixels×4320 vertical pixels), or square 4K (3840 or more horizontal pixels×3840 or more vertical pixels). The light receiving sensor 50054 may be one sensor chip, or a plurality of sensor chips. For example, a prism may be provided to separate the incident light into predetermined wavelength bands, and the wavelength bands may be imaged by different light receiving sensors. A plurality of light receiving sensors may be provided for stereoscopic viewing. The light receiving sensor 50054 may be a sensor having a chip structure including an arithmetic processing circuit for image processing, or may be a sensor for time of flight (ToF). The transmission system is, for example, an optical fiber cable system or a wireless transmission system. The wireless transmission only needs to be capable of transmitting the pixel signal generated by the endoscope 5001, and, for example, the endoscope 5001 may be wirelessly connected to the CCU 5039, or the endoscope 5001 may be connected to the CCU 5039 via a base station in an operating room. At this time, the endoscope 5001 may transmit not only the pixel signal, but also simultaneously information (for example, a processing priority of the pixel signal and/or a synchronization signal) related to the pixel signal. In the endoscope, the scope may be integrated with the camera, and the light receiving sensor may be provided at the distal end of the scope.

### (Camera Control Unit (CCU))

The CCU 5039 is a control device for controlling the endoscope 5001 and the light source device 5043 connected to the CCU 5039 in an integrated manner, and is, for example, as illustrated in FIG. 2, an image processing device including a field-programmable gate array (FPGA) 50391, a central processing unit (CPU) 50392, a random access memory 50393, a read-only memory (ROM) 50394, a graphics processing unit (GPU) 50395, and an interface (I/F) 50396. The CCU 5039 may control the display device 5041, the recording device 5053, and the output device 5055 connected to the CCU 5039 in an integrated manner. The CCU 5039 controls, for example, irradiation timing, irradiation intensity, and a type of an irradiation light source of the light source device 5043. The CCU 5039 also performs image processing, such as development processing (for example, demosaic processing) and correction processing, on the pixel signal output from the endoscope 5001, and outputs the processed image signal (for example, an image) to an external device such as the display device 5041. The CCU 5039 also transmits a control signal to the endoscope 5001 to control driving of the endoscope 5001. The control signal is information on an imaging condition such as a magnification or the focal length of the imaging section. The CCU 5039 may have a function to down-convert the image, and may be configured to be capable of simultaneously outputting a higher-resolution (for example, 4K) image to the display device 5041 and a lower-resolution (for example, high-definition (HD)) image to the recording device 5053.

The CCU 5039 may be connected to external equipment (such as a recording device, a display device, an output device, and a support device) via an IP converter for converting the signal into a predetermined communication protocol (such as the Internet Protocol (IP)). The connection between the IP converter and the external equipment may be established using a wired network, or a part or the whole of the network may be established using a wireless network. For example, the IP converter on the CCU 5039 side may have a wireless communication function, and may transmit the received image to an IP switcher or an output side IP converter via a wireless communication network, such as the fifth-generation mobile communication system (5G) or the sixth-generation mobile communication system (6G).

### (Light Source Device)

The light source device 5043 is a device capable of emitting the light having predetermined wavelength bands, and includes, for example, a plurality of light sources and a light source optical system for guiding the light of the light sources. The light sources are, for example, xenon lamps, light-emitting diode (LED) light sources, or laser diode (LD) light sources. The light source device 5043 includes, for example, the LED light sources corresponding to three respective primary colors of red (R), green (G), and blue (B), and controls output intensity and output timing of each of the light sources to emit white light. The light source device 5043 may include a light source capable of emitting special light used for special light observation, in addition to the light sources for emitting normal light for normal light observation. The special light is light having a predetermined wavelength band different from that of the normal light being light for the normal light observation, and is, for example, near-infrared light (light having a wavelength of 760 nm or longer), infrared light, blue light, or ultraviolet light. The normal light is, for example, the white light or green light. In narrow band imaging that is a kind of special light observation, blue light and green light are alternately emitted, and thus the narrow band imaging can image a predetermined tissue such as a blood vessel in a mucosal surface at high contrast using wavelength dependence of light absorption in the tissue of the body. In fluorescence observation that is a kind of special light observation, excitation light is emitted for exciting an agent injected into the tissue of the body, and fluorescence emitted by the tissue of the body or the agent as a label is received to obtain a fluorescent image, and thus the fluorescence observation can facilitate the operator to view, for example, the tissue of the body that is difficult to be viewed by the operator with the normal light. For example, in fluorescence observation using the infrared light, the infrared light having an excitation wavelength band is emitted to an agent, such as indocyanine green (ICG), injected into the tissue of the body, and the fluorescence light from the agent is received, whereby the fluorescence observation can facilitate viewing of a structure and an affected part of the tissue of the body. In the fluorescence observation, an agent (such as 5-aminolevulinic acid (5-ALA)) may be used that emits fluorescence in a red wavelength band by being excited by the special light in a blue wavelength band. The type of the irradiation light of the light source device 5043 is set by control of the CCU 5039. The CCU 5039 may have a mode of controlling the light source device 5043 and the endoscope 5001 to alternately perform the normal light observation and the special light observation. At this time, information based on a pixel signal obtained by the special light observation is preferably superimposed on a pixel signal obtained by the normal light observation. The special light observation may be an infrared light observation to observe a site inside the surface of an organ and a multi-spectrum observation utilizing hyperspectral spectroscopy. A photodynamic therapy may be incorporated.

### (Recording Device)

The recording device 5053 is a device for recording the pixel signal (for example, an image) acquired from the CCU 5039, and is, for example, a recorder. The recording device 5053 records an image acquired from the CCU 5039 in a hard disk drive (HDD), a Super Density Disc (SDD), and/or an optical disc. The recording device 5053 may be connected to a network in a hospital to be accessible from equipment outside the operating room. The recording device 5053 may have a down-convert function or an up-convert function.

### (Display Device)

The display device 5041 is a device capable of displaying the image, and is, for example, a display monitor. The display device 5041 displays a display image based on the pixel signal acquired from the CCU 5039. The display device 5041 may include a camera and a microphone to function as an input device that allows instruction input through gaze recognition, voice recognition, and gesture.

### (Output Device)

The output device 5055 is a device for outputting the information acquired from the CCU 5039, and is, for example, a printer. The output device 5055 prints, for example, a print image based on the pixel signal acquired from the CCU 5039 on a sheet of paper.

### (Support Device)

The support device 5027 is an articulated arm including a base 5029 including an arm control device 5045, an arm 5031 extending from the base 5029, and a holding part 5032 mounted at a distal end of the arm 5031. The arm control device 5045 includes a processor such as a CPU, and operates according to a predetermined computer program to control driving of the arm 5031. The support device 5027 uses the arm control device 5045 to control parameters including, for example, lengths of links 5035 constituting the arm 5031 and rotation angles and torque of joints 5033 so as to control, for example, the position and attitude of the endoscope 5001 held by the holding part 5032. This control can change the position or attitude of the endoscope 5001 to a desired position or attitude, makes it possible to insert the scope 5003 into the patient 5071, and can change the observed area in the body. The support device 5027 functions as an endoscope support arm for supporting the endoscope 5001 during the operation. Thus, the support device 5027 can play a role of a scopist who is an assistant holding the endoscope 5001. The support device 5027 may be a device for holding a microscope device 5301 to be described later, and can be called a medical support arm. The support device 5027 may be controlled using an autonomous control method by the arm control device 5045, or may be controlled using a control method in which the arm control device 5045 performs the control based on input of a user. The control method may be, for example, a master-slave method in which the support device 5027 serving as a slave device (replica device) that is a patient cart is controlled based on a movement of a master device (primary device) that is an operator console at a hand of the user. The support device 5027 may be remotely controllable from outside the operating room.

The example of the endoscope system 5000 to which the technology according to the present disclosure is applicable has been described above. For example, the technology according to the present disclosure may be applied to a microscope system.

### (Microscope System)

FIG. 3 is a diagram illustrating an example of a schematic configuration of a microscopic surgery system to which the technology according to the present disclosure is applicable. In the following description, the same components as those of the endoscope system 5000 will be denoted by the same reference numerals, and the description thereof will not be repeated.

FIG. 3 schematically illustrates a situation where the operator 5067 performs an operation on the patient 5071 on the patient bed 5069 using a microscopic surgery system 5300. For the sake of simplicity, FIG. 3 does not illustrate a cart 5037 among the components of the microscopic surgery system 5300, and illustrates the microscope device 5301 instead of the endoscope 5001 in a simplified manner. The microscope device 5301 may refer to a microscope 5303 provided at the distal end of the links 5035, or may refer to the overall configuration including the microscope 5303 and the support device 5027.

As illustrated in FIG. 3, during the operation, the microscopic surgery system 5300 is used to display an image of a surgical site captured by the microscope device 5301 in a magnified manner on the display device 5041 installed in the operating room. The display device 5041 is installed in a position facing the operator 5067, and the operator 5067 performs various procedures, such as excision of an affected part, on the surgical site while observing the state of the surgical site using the image displayed on the display device 5041. The microscopic surgery system is used in, for example, ophthalmic operation and neurosurgical operation.

The respective examples of the endoscope system 5000 and the microscopic surgery system 5300 to which the technology according to the present disclosure is applicable have been described above. Systems to which the technology according to the present disclosure is applicable are not limited to such examples. For example, the support device 5027 can support, at the distal end thereof, another observation device or another surgical tool instead of the endoscope 5001 or the microscope 5303. Examples of the other applicable observation device include forceps, tweezers, a pneumoperitoneum tube for pneumoperitoneum, and an energy treatment tool for incising a tissue or sealing a blood vessel by cauterization. By using the support device to support the observation device or the surgical tool described above, the position thereof can be more stably fixed and the load of the medical staff can be lower than in a case where the medical staff manually supports the observation device or the surgical tool. The technology according to the present disclosure may be applied to a support device for supporting such a component other than the microscope.

The technology according to the present disclosure can be suitably applied to the light source device 5043 among the components explained above. Specifically, the technology according to the present disclosure is suitably used in a configuration in which the light source device 5043 simultaneously irradiates normal light and special light. By applying the technology according to the present disclosure, it is possible to suppress dependency of a light amount ratio of special light by a narrowband light source and normal light by a wideband light source on a light guide diameter and it is possible to configure the light source device 5043 to be smaller. For that reason, observation of a surgical site by the endoscope is easier, surgery can be more safely and reliably performed, and a space in the operating room can be more effectively used.

### 2. Existing Technology

Before the embodiments of the present disclosure are explained, an existing technology is explained.

As an apparatus for viewing an internal structure of an object, an endoscope has been widely used. In particular, in the medical field, according to the development of the surgical technique, the endoscope has rapidly spread and is now indispensable in many medical fields. In an endoscope device in the existing technology, only a white light source such as a lamp light source (a xenon lamp, a halogen lamp, or the like) or a light emitting diode (LED) light source is mounted as a light source for illuminating an affected part in both a flexible endoscope and a rigid endoscope.

On the other hand, in recent years, a function of performing fluorescence observation of a drug has been added to an endoscope. The endoscope has evolved from equipment that simply observes an affected part to equipment that supports a technique of a doctor.

The fluorescence observation of a drug refers to observing fluorescent light generated in response to certain light (excitation light). A part of drugs has been already covered by insurance and widely spread. The drug has an intrinsic absorption spectrum and, when excited by light having the same wavelength as the peak wavelength of the absorption spectrum, can most efficiently emit fluorescent light.

According to the technological progress on a camera imager side and a processor side, it is possible to superimpose an affected part image obtained by a white light source spectrum and a lesion image obtained by fluorescence observation by simultaneously turning on the white light source as well in the case of the fluorescence observation. Accordingly, by displaying a lesion in real time and at a more accurate position, it is possible to realize advanced surgical support for doctors.

Such an endoscope light source capable of simultaneously irradiating excitation light and white light includes one or more excitation light sources and one or more white light sources and includes a mechanism for multiplexing and emitting rays emitted from these two or more light sources with optical systems inside the light sources.

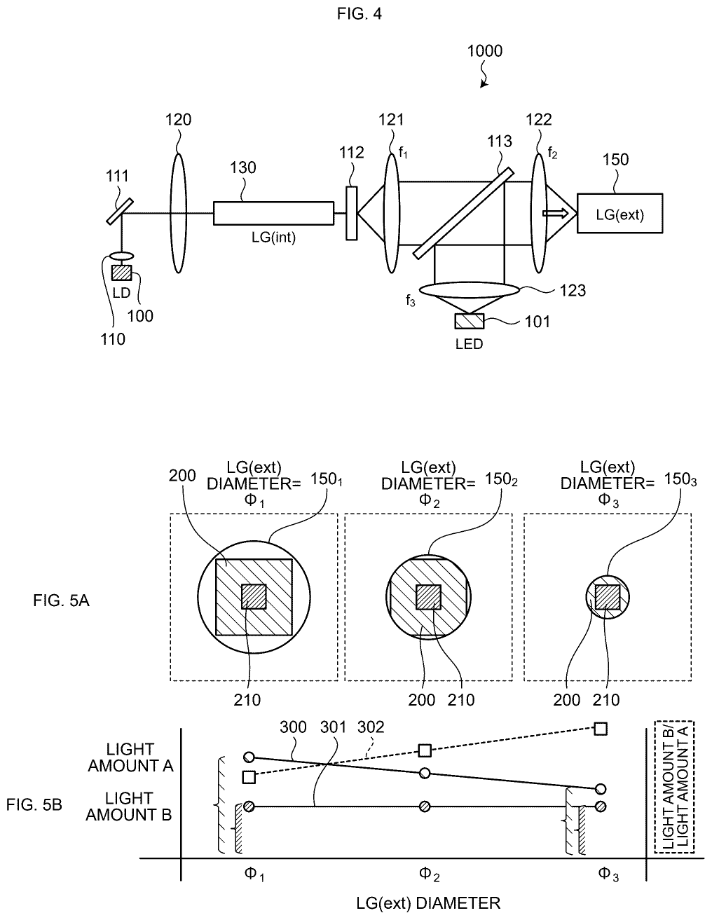

FIG. 4 is a schematic diagram illustrating a configuration of an example of a light source device capable of simultaneously irradiating excitation light and white light according to the existing technology. In FIG. 4, the light source device 1000 includes light sources 100 and 101, a collimate lens 110, a total reflection mirror 111, a diffusion plate 112, and a multiplexer 113, lenses 120 to 123, an internal light guide 130, and an external light guide 150. Note that the focal lengths of the lenses 121, 122, and 123 are respectively represented as focal lengths f₁, f₂, and f₃.

The light source 100 uses, for example, a laser diode as a light emitting element. The light source 100 is a narrowband light source that emits and narrowband light serving as excitation light with the laser diode. The light source 101 uses, for example, a light emitting diode (LED) as a light emitting element. The light source 101 is a wideband light source that emits, for example, wideband light serving as white light with the LED. Here, the narrowband light is, for example, light in a wavelength band based on a single wavelength and the wideband light is, for example, light in a wavelength band including a visible light wavelength region. Not only this, but the wideband light may be light in a wavelength band based on a plurality of single wavelengths having different wavelengths.

The narrowband light emitted from the light source 100 is changed to collimated light by the collimate lens 110, totally reflected by the total reflection mirror 111 to change an optical path, and made incident on an incident end of the internal light guide 130 via the lens 120. The internal light guide 130 is illustrated as LG (int) as well in the figure.

The internal light guide 130 is, for example, a rod integrator having a prismatic shape and repeatedly totally reflects incident light on an inner wall to uniformize a light amount distribution at an emission end. The narrowband light having the uniformized light amount distribution in the internal light guide 130 is emitted from the emission end of the internal light guide 130 as secondary light source light, diffused by the diffusion plate 112, and made incident on the lens 121. The secondary light source light emitted from the lens 121 is made incident on a first incident unit of the multiplexer 113.

On the other hand, the wideband light emitted from the light source 101 is made incident on a second incident unit of the multiplexer 113 via the lens 123.

The multiplexer 113 multiplexes the light made incident on the first incident unit and the light made incident on the second incident unit and emits the light as multiplexed light. The multiplexer 113 may be configured using, for example, a bandpass filter that transmits light in a wavelength band of narrowband light and reflects light in other wavelength bands. In this case, the secondary light source light based on the narrowband light emitted from the internal light guide 130 is transmitted through the multiplexer 113 and the wideband light emitted from the light source 101 is reflected by the multiplexer 113 and an optical path of the wideband light is changed to coincide with an optical path of the narrowband light. Accordingly, the narrowband light (the secondary light source light) and the wideband light are multiplexed.

The multiplexed light emitted from the multiplexer 113 is made incident on the external light guide 150 via, for example, the lens 122, which is a condenser lens. The external light guide 150 is, for example, an optical fiber bundle and transmits light incident on one end to the other end and emits the light. An end of the external light guide 150 from which the multiplexed light is emitted is inserted into the body of the patient 5071, for example, together with the scope 5003 or while being included in the scope 5003. As the external light guide 150, an appropriate light guide is selected as appropriate according to a use, a use method, and the like of the external light guide 150 and is replaced and used.

The optical configuration of the light source according to the existing technology illustrated in FIG. 4 has the following problems.

As illustrated in FIG. 4, in the light source device 1000, the light source 100, which is the narrowband light source, uses the laser diode (LD) as the light emitting element and the light source 101, which is the wideband light source, uses the LED as the light emitting element. In this configuration, as a result of designing an imaging size of the LD light at an incident end (an incident surface) of the internal light guide 130 to be small using the characteristics of the LD, an imaging size of the LD light and an imaging size of the LED light at an incident end (an incident surface) of the external light guide 150 are sometimes different. When the external light guides 150 having various diameters are attached to the light source device 1000 in this state, a ratio of a light amount A of wideband light (LED light) taken into the external light guides 150 and a light amount B of narrowband light (LD light) taken into the external light guides 150 changes for each of the diameters of the external light guides 150.

FIG. 5 is a schematic diagram illustrating an example of a relation between a diameter of the external light guide 150 and a ratio of light amounts of narrowband light and wideband light according to the existing technology. In FIG. 5, a section (a) schematically illustrates a relation between external light guides 150₁, 150₂, and 150₃ having different diameters Φ and a light amount distribution 200 and a light amount distribution 210 of wideband light and narrowband light (secondary light source light) respectively taken into the external light guides 150₁, 150₂, and 150₃. Here, it is assumed that diameters Φ₁, Φ₂, and Φ₃ of the external light guides 150₁, 150₂, and 150₃ have a relation of Φ₁>Φ₂>Φ₃.

A section (b) in FIG. 5 illustrates light amounts A and B respectively corresponding to the light amount distributions 200 and 210 and light amount ratios of the light amount A and the light amount B in the external light guides 150₁, 150₂, and 150₃. In the section (b) of FIG. 5, a characteristic line 300 indicates the light amount A, a characteristic line 301 indicates the light amount B, and a characteristic line 302 indicates a light amount ratio (the light amount B/the light amount A) for the external light guides 150₁ to 150₃.

In the internal light guide 130, for example, when the number of times of reflection of light on the inside is not sufficient and a light intensity distribution (Near Field Pattern: NFP) is not sufficiently uniformized at an output end, the light amount distribution 210 of the secondary light source light concentrates on the center at an emission end of the internal light guide 130. In this case, the secondary light source light is emitted from the internal light guide 130 to concentrate on a narrow range. For that reason, the secondary light source light emitted from the internal light guide 130 and incident on the external light guides 150₁ to 150₃ fits within a range of the diameters Φ₁ to Φ₃ in all the external light guides 150₁ to 150₃ having the diameters Φ₁ to Φ₃. The light amount B of the incident light is made constant as indicated by the characteristic line 301.

On the other hand, in the wideband light, vignetting occurs according to the diameters Φ₁ to Φ₃ of the external light guides 150₁ to 150₃. For that reason, in the wideband light, the light amount A of the incident light changes according to the diameters Φ₁ to Φ₃ as indicated by the characteristic line 300.

For example, it is conceived that the light amount distribution 200 of the wideband light is designed to be maximum at an assumed maximum diameter Φ (the diameter Φ₁ in this example) of the external light guide 150. In this case, as the diameter Φ of the external light guide 150 decreases with respect to the maximum diameter Φ, light is not taken into the external light guide 150 from a peripheral portion of the light amount distribution 200.

Therefore, the light amount ratio=the light amount B/the light amount A changes depending on the diameters Φ₁ to Φ₃ of the external light guides 150₁ to 150₃ as indicated by the characteristic line 302.

When the light amount ratio of the narrowband light and the wideband light changes according to the diameter Φ of the external light guide 150 as explained above, the brightness of the narrowband light and the wideband light at the time of fluorescence observation changes, This affects observation image quality.

On the other hand, since it is known that the light amount ratio of the narrowband light and the wideband light changes according to the diameter of the external light guide 150, it is also conceivable to estimate a change in the light amount ratio and set the change as an image quality parameter. However, in this method, it is necessary to set the image quality parameter at the time of the fluorescence observation for each diameter Φ of the external light guide 150. This increases the number of development steps on a design side and time and effort of setting at the time of observation on a user side.

In order to avoid the change in the light amount ratio explained above of the narrowband light and the wideband light made incident on the external light guide 150 due to the diameter Φ of the external light guide 150, there has also been proposed a method of providing a transmission rod between an optical path last lens and the external light guide 150 to multiplex the narrowband light and the wideband light (for example, JP 2012-509098 W).

However, since a ray emitted from the terminal end of the transmission rod diverges, when the external light guide 150 is provided behind the terminal end of the transmission rod, light coupling efficiency is deteriorated and optical efficiency of light finally reaching an endoscope distal end is deteriorated.

### 3. Overview of the Embodiments of the Present Disclosure

Subsequently, an overview of the embodiments of the present disclosure is explained.

In order to suppress the dependency described above of the light amount ratio of the narrowband light and the wideband light made incident on the external light guide 150 on the diameter Φ of the external light guide 150, it is necessary to set the sizes of the narrowband light and the wideband light imaged at the incident end of the external light guide 150 to be equal to or larger than the maximum diameter of the external light guide 150 used by the user. From the viewpoint of optical efficiency, it is desired to set imaging sizes of the narrowband light and the wideband light at the incident end of the external light guide 150 to the same size.

That is, it is preferable to design the units such that optical characteristics relating to the narrowband light and optical characteristics relating to the wideband light satisfy the following Expression (1).

$$0. 67 \times {D}_{A} / {f}_{3} \leq {D}_{rod} / {f}_{1} \leq 1.33 \times {D}_{A} / {f}_{3} \qquad ( 1 )$$

Note that, in Expression (1), D_{rod} represents a size (hereinafter, diameter D_{rod}) of the emission end (the emission surface) of the internal light guide 130 and D_{A} represents a size (hereinafter, diameter D_{A}) of a light emitting surface of the light source 101 that emits the wideband light. Note that, when the shapes of the surfaces are rectangular, the diameter D_{rod} and the diameter D_{A} respectively indicate diagonal lengths of a rectangle. In addition, f₁ represents a focal length (hereinafter, focal length f₁) of the lens 121 on which the narrowband light emitted from the internal light guide 130 and diffused by the diffusion plate 112 is made incident and f₃ represents a focal length (hereinafter, focal length f₃) of the lens 123 on which the wideband light emitted from the light source 101 is made incident.

The light emitted from the internal light guide 130 is imaged on the lens 120 with the size in the lens 122. The light emitted from the light source 101 is imaged on the lens 123 with the size in the lens 122. Expression (1) described above means that the sizes of the light respectively imaged on the lens 122 coincide with each other. In other words, Expression (1) can be considered equalizing an apparent light source size on the emission surface of the lens 120 and an apparent light source size on the emission surface of the lens 123.

It is more preferable to change the coefficients in the Expression (1) described above and design the units to satisfy the following Expression (2).

$$0. 9 \times {D}_{A} / {f}_{3} \leq {D}_{rod} / {f}_{1} \leq 1.1 \times {D}_{A} / {f}_{3} \qquad ( 2 )$$

Note that the coefficients of Expressions (1) and (2) described above are examples and are not limited to the examples. The coefficients of Expressions (1) and (2) gives margins to ideal conditions concerning the optical characteristic relating to the narrowband light and the optical characteristic relating to the wideband light indicated by the following Expression (3).

$${D}_{rod} / {f}_{1} = {D}_{A} / {f}_{3} \qquad ( 3 )$$

In practice, the diameter D_{A} of the light source 101 using the LED as the light emitting element is often already determined as device specifications. Design flexibility of the diameter D_{A} is low. For that reason, the diameter D_{rod} and the focal lengths f₁ and f₃ are designed as parameters.

On the other hand, in this light source optical system, it is necessary to uniformize an NFP at the emission end of the internal light guide 130. For example, it is preferable to set the number of times of reflection in the internal light guide 130 to three to six or more as a guide. For this reason, when the diameter D_{Rod} is increased, length L of the entire length of the internal light guide 130 also needs to be increased by a scale multiple of the size increase of the diameter D_{rod} in order to maintain the number of times of reflection.

For example, when the diameter D_{rod} is increased from 1 mm to 2 mm, the length L also needs to be doubled. In this case, it is likely that the size of the light source device 5043 increases.

In general, when a rod width D (corresponding to the width of the internal light guide 130), a total rod length L (corresponding to the total length of the internal light guide 130), a glass material refractive index n, a numerical aperture NA with respect to an incident ray, and the number of times of reflection R are set, a parameter relation is expressed by the following Expression (4).

$$R = L / D \times tan \{ {sin}^{- 1} ( NA / n ) \} \qquad ( 4 )$$

As it is seen from Expression (4), in order to maintain the number of times of reflection R, when the rod width D is increased, it is necessary to increase the total rod length L at the same ratio. On the other hand, when the total rod length L increases, the total length of the internal light guide 130 increases. This makes it difficult to maintain component quality or causes an increase in cost. Since the entire length of the optical system also increases, the overall cost of the light source device 5043 is also likely to increase.

An embodiment of the present disclosure proposes an optical configuration capable of suppressing dependency of a light amount ratio of narrowband light and wideband light on the diameter of the external light guide 150 while solving the parameter constraint of the optical system explained above.

### 4. First Embodiment of the Present Disclosure

Subsequently, a first embodiment of the present disclosure is explained.

In the first embodiment of the present disclosure, in order to uniformize an NFP at the output end of the internal light guide 130, that is, maintain the number of times of reflection of light in the internal light guide 130 at a predetermined number of times or more, it is also a measure to control the numerical aperture NA in addition to controlling the total rod length L and the rod width D based on Expression (4) described above. In the first embodiment, a diffusion plate is provided in front of the internal light guide 130 as means for controlling the numerical aperture NA.

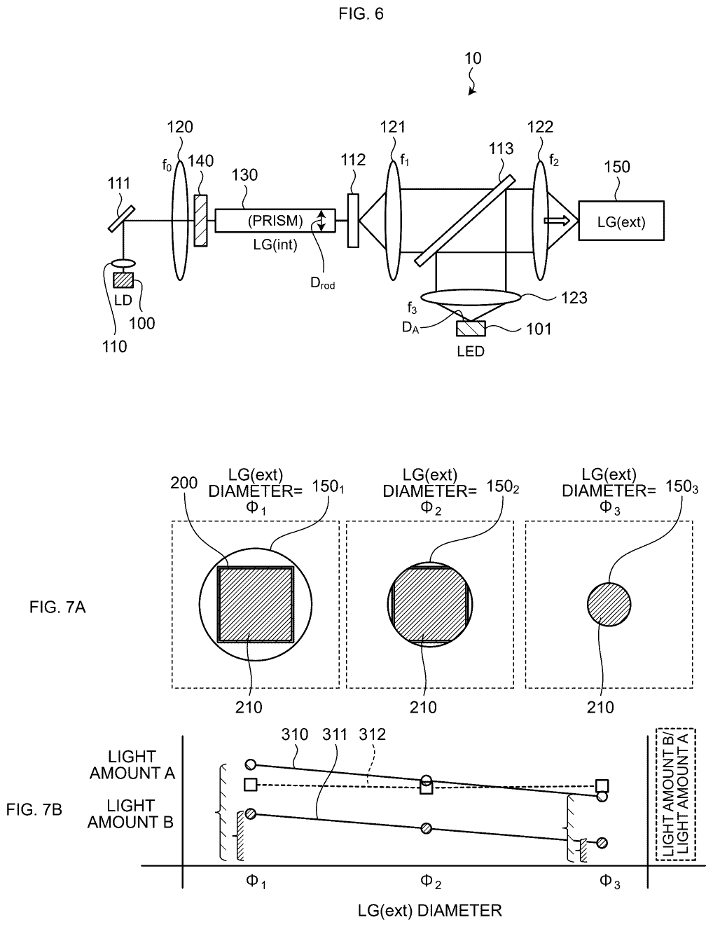

FIG. 6 is a schematic diagram illustrating a configuration of an example of a light source device according to the first embodiment; In FIG. 6, the light source device 10 is different from the light source device 1000 according to the existing technology explained with reference to FIG. 4 in that a diffusion plate 140 is added between the lens 120 and the internal light guide 130. The diffusion plate 140 diffuses and emits incident light at a diffusion angle α.

In the following explanation, when the diffusion plate 12 disposed on the emission end side of the internal light guide 130 is set a main diffusion plate as appropriate, the diffusion plate 140 disposed on the incident end side of the internal light guide 130 can be referred to as pre-diffusion plate. In the following explanation, the diffusion plate 140 is referred to as pre-diffusion plate 140.

The pre-diffusion plate 140 functions as a conversion element that converts collimated light emitted from the light source 100 via the lens 120 into light diffused at the predetermined diffusion angle α. As the pre-diffusion plate 140, a fly-eye lens or a micro-lens array can be applied. As the pre-diffusion plate 140, a member like a so-called polished glass in which irregularities are formed at random on the surface of glass by chemical treatment, sand, or the like may be used.

The collimated light emitted from the lens 120 is diffused by the pre-diffusion plate 140 at the predetermined diffusion angle and is made incident on the internal light guide 130. A range of the predetermined diffusion angle α at which the pre-diffusion plate 140 diffuses the incident light is determined based on factors such as light condensing efficiency of the light emitted from the light source 100 on the incident surface of the internal light guide 130, a total reflection condition in the internal light guide 130, and the number of times of reflection in the internal light guide 130. Within the range, design only has to be performed from the viewpoint of cost, efficiency, and the like. The predetermined diffusion angle α is preferably set to an angle at which diffused light does not exceed a range of a surface (an incident surface) at the incident end of the internal light guide 130.

Note that reducing a focal length f₀ of the lens 120 disposed in the front of the pre-diffusion plate 140 is also one of means for controlling the numerical aperture NA. However, when the focal length f₀ of the lens 120 is reduced, the sensitivity of an optical system from the light source 100 to the internal light guide 130 increases and design robustness decreases. For that reason, in the first embodiment, by disposing the pre-diffusion plate 140 between the internal light guide 130 and the lens 120 and setting the diffusion angle α of the pre-diffusion plate 140 to a predetermined value to make it possible to maintain the number of times of reflection of a ray inside the internal light guide 130 at a predetermined number of times or more.

As the diffusion plate, a diffusion plate having distributions with different divergence angles in two axial directions besides a general Gaussian uniform divergence angle distribution and a diffusion plate having a top-hat distribution are also distributed in the market according to the progress of a machining process. Besides the examples explained above, by appropriately selecting these diffusion plates as the pre-diffusion plate 140, both of the optical efficiency and the number of times of reflection can be optimized.

FIG. 7 is a schematic diagram illustrating an example of a relation between a diameter of the external light guide 150 and a ratio of light amounts of narrowband light and wideband light according to the first embodiment. Meanings and the like of the units illustrated in FIG. 7 are the same as those in FIG. 5 explained above. Therefore, explanation of the meanings and the like is omitted here.

In the first embodiment, the number of times of reflection of light on the inside of the internal light guide 130 can be maintained at a predetermined number of times or more by using the pre-diffusion plate 140. An NFP at the output end of the internal light guide 130 is sufficiently uniformized. For that reason, it is possible to further increase the range of the light amount distribution 210 of the secondary light source light emitted from the internal light guide 130.

That is, in the first embodiment, as explained using Expression (1) and the like, the size of the secondary light source light emitted from the internal light guide 130 via the lens 120 and the size of the wideband light emitted from the light source 101 via the lens 123, the secondary light source light and the wideband light being respectively imaged on the lens 122, are matched with each other. For that reason, as illustrated in a section (a) of FIG. 7, the light amount distribution 210 by the secondary light source light emitted from the internal light guide 130 can be substantially matched with the light amount distribution 200 of the wideband light by the light source 101 designed to be maximum at the assumed maximum diameter Φ (the diameter Φ₁ in this example) of the external light guide 150.

In the external light guides 150₂ and 150₃ having the smaller diameter Φ, the diameter Φ decreases while the light amount distributions 200 and 210 in the external light guide 150₁ are maintained.

In the section (a) of FIG. 7, at the diameters Φ₂ and Φ₃, since the light amount distribution 200 and the light amount distribution 210 substantially overlap, description of the light amount distribution 200 is omitted.

Like the section (b) of FIG. 5, a section (b) of FIG. 7 illustrates the light amounts A and B respectively corresponding to the light amount distributions 200 and 210 and the light amount ratios of the light amount A and the light amount B in the external light guides 150₁, 150₂, and 150₃. In the section (b) of FIG. 7, a characteristic line 310 indicates a light amount A, a characteristic line 311 indicates a light amount B, and a characteristic line 312 indicates a light amount ratio (the light amount B/the light amount A) for the external light guides 150₁ to 150₃.

Since the light amount distributions 200 and 201 of the light amounts A and B coincide, the light amounts A and B respectively change according to the diameters Φ₁ to Φ₃ of the external light guides 150₁ to 150₃ as indicated by the characteristic lines 310 and 311 in the section (b) of FIG. 7. Therefore, the light amount ratio=the light amount B/the light amount A is substantially constant without depending on the diameters Φ₁ to Φ₃ of the external light guides 150₁ to 150₃ as indicated by the characteristic line 312.

As explained above, in the first embodiment, the dependency of the light amount ratio of the narrowband light and the wideband light on the diameter Φ of the external light guide 150 is suppressed. Therefore, it is possible to obtain satisfactory observation image quality even when the external light guide 150 is replaced with a light guide having a different diameter.

Note that it is preferable that at least one of the size (the diameter D_{rod}) of the emission end of the internal light guide 130 and the size (the diameter D_{A}) of the light emitting surface of the light source 101 is smaller than the size of the incident end of the external light guide 150 because the narrowband light and the wideband light can be more efficiently taken in by the external light guide 150. For example, when the size of the incident end of the external light guide 150 is represented as LG_{SIZE}, the diameter D_{rod}, the diameter D_{A}, and LG_{SIZE} are set to satisfy the following Expression (5). A symbol “V” indicates a logical sum.

$$( {D}_{rod} \leq L {G}_{SIZE} ) ∨ ( {D}_{A} \leq L {G}_{SIZE} ) \qquad ( 5 )$$

As explained above, in the first embodiment of the present disclosure, the pre-diffusion plate 140 having the diffusion angle α is disposed in the vicinity of the incident end side of the internal light guide 130 and the numerical aperture NA of the internal light guide 130 with respect to the incident ray is increased. Accordingly, it is possible to select a size of the light emitting surface of the light source 101 and perform lens design such that the relation illustrated in Expression (1) described above is established while not increasing the total length L of the internal light guide 130 and while controlling the diameter D_{rod} of the emission end (the emission surface) of the internal light guide 130. That is, an imaging size of the secondary light source light emitted from the internal light guide 130 and an imaging size of the wideband light emitted from the light source 101 at the incident end of the external light guide 150 can be set substantially the same.

Note that, in the above explanation, as the types of the light sources 100 and 101, the light source 100 is the narrowband light source (the laser diode) and the light source 101 is the wideband light source (the LED). However, the types of the light sources 100 and 101 are not limited to this example. For example, both of the light sources 100 and 101 may be narrowband light sources or may be wideband light sources.

In the example illustrated in FIG. 6, each of the lenses 120 to 123 is configured from one lens. However, the lenses 120 to 123 are not limited to this example. A part or all of the lenses 120 to 123 may be a collective lens including a plurality of lenses.

Further, in the example illustrated in FIG. 6, the wideband light emitted from the light source 101 is illustrated as being directly made incident on the lens 123. However, but the wideband light is not limited to this example. For example, a diffusion plate may be disposed between the light source 101 and the lens 123.

Furthermore, in the above explanation, it is explained that the light source that emits the narrowband light made incident on the internal light guide 130 is only one light source 100. However, the light source is not limited to this example. That is, the light source device 10 according to the first embodiment may include a plurality of light sources that emit light made incident on the internal light guide 130.

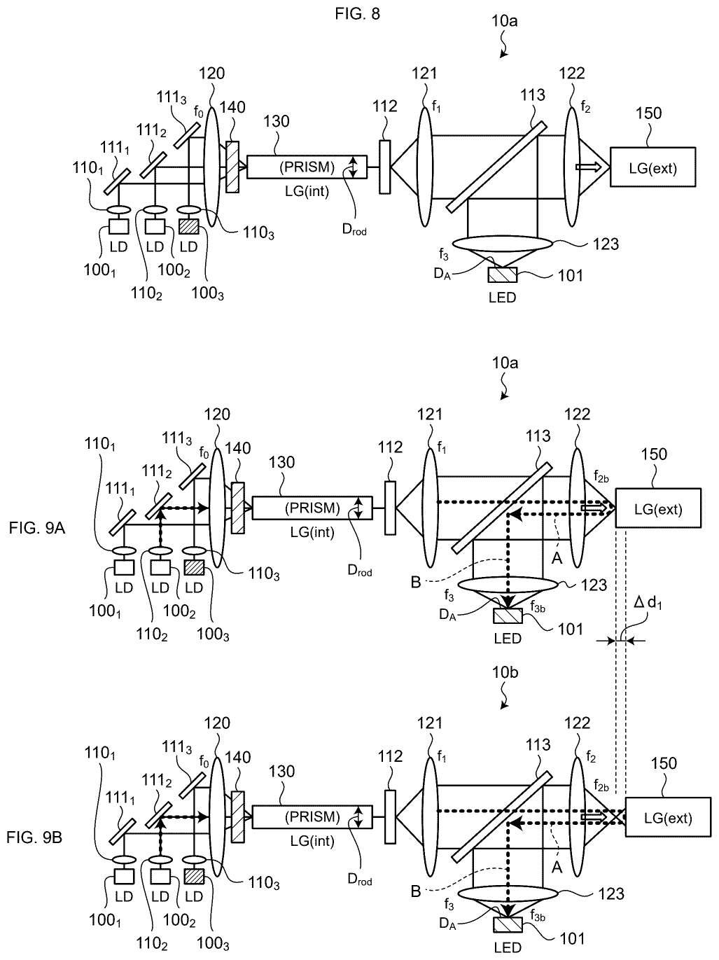

FIG. 8 is a schematic diagram illustrating an example of a light source device corresponding to a plurality of light sources that respectively emit narrowband light according to the first embodiment. In FIG. 8, a light source device 10a includes a plurality of light sources 100₁, 100₂, and 100₃ that respectively emit narrowband light. Light emitted from the light sources 100₁ to 100₃ is respectively emitted to total reflection mirrors 111₁ to 111₃ via collimate lenses 110₁ to 110₃. Optical paths of the light are respectively changed by the total reflection mirrors 111₁ to 111₃ and the light is made incident on the pre-diffusion plate 140 via the lens 120.

Even in such a configuration, the narrowband light emitted from the light sources 100₁ to 100₃ is diffused at the diffusion angle α in the pre-diffusion plate 140 and is made incident on the internal light guide 130. Therefore, the narrowband light is changed to secondary light source light, an NFP of which is sufficiently uniformized at the output end of the internal light guide 130, is multiplexed with the wideband light emitted from the light source 101, and is made incident on the external light guide 150.

### 5. Second Embodiment of the Present Disclosure

Subsequently, a second embodiment of the present disclosure is explained. The second embodiment is an example in which, in the light source device 10 or 10a in the first embodiment explained above, irradiation of the light source 101, which is an LED, with multiplexed light reflected at the incident end of the external light guide 150 is suppressed.

FIG. 9 is a schematic diagram illustrating a configuration of an example of a light source device according to the second embodiment. In FIG. 9, a section (a) illustrates the light source device 10a equivalent to the light source device 10a illustrated in FIG. 8 and the light sources 100₁, 100₂, and 100₃ that respectively emit rays having different wavelengths are provided on the internal light guide 130 side.

As indicated by an arrow A in the section (a) of FIG. 9, after the rays emitted from the light sources 100₁ to 100₃ reach the external light guide 150, there is a component reflected from the surface or the inside of the external light guide 150. Further, as indicated by an arrow B in the figure, in some case, the reflected component is reflected by the multiplexer 113, an optical path of the reflected component is changed, and the light source 101 is irradiated with the reflected component. At that time, it is assumed that the light source 101 is an LED, the light sources 100₁ to 100₃ are respectively LDs or LEDs, and at least one of the light sources 100₁ to 100₃ includes an excitation wavelength spectrum component of the light source 101.

In this case, the light source 101 emits, as excitation light, light emitted from the light sources 100₁ to 100₃ and returned from the external light guide 150 (hereinafter referred to as return light as appropriate). Light emitted from the light source 101 that emits light in response to the return light from the external light guide 150 is reflected by the multiplexer 113 and reaches the incident surface of the external light guide 150 as at the time when the light source 101 originally emits light. That is, for example, even when only at least one of the light sources 100₁ to 100₃ emits light and the light source 101 does not emit light, the external light guide 150 is irradiated with the light including a component of the light emitted from the light source 101.

In order to avoid a situation in which the light source 101 is excited by the return light from the external light guide 150 to emit light, it is conceivable to design, in a bandpass filter configuring the multiplexer 113, the reflectance of light having a wavelength including an excitation wavelength of the light source 101 to be low and suppress reflection of the return light from the external light guide 150 to the light source 101 side.

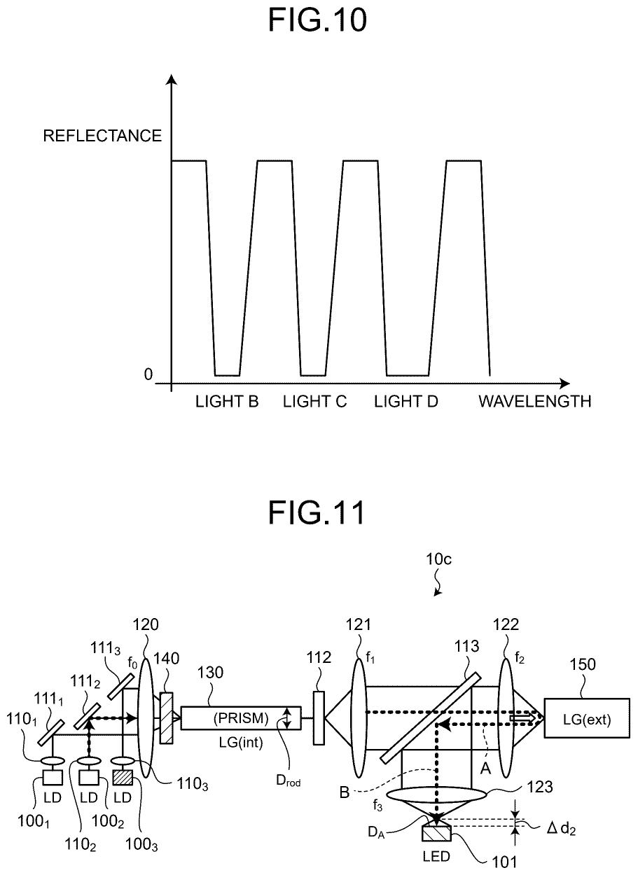

FIG. 10 is a schematic diagram illustrating an example of a reflection characteristic of the bandpass filter in the case in which the reflectance of the light having the wavelength including the excitation wavelength of the light source 101 is designed to be low. In FIG. 10, light B, light C, and light D respectively indicate light emitted by the light sources 100₁ to 100₃. As explained above, in the bandpass filter configuring the multiplexer 113, the reflectance of a wavelength region of the light emitted by the light sources 100₁ to 100₃ is designed to be low. However, it is difficult to reduce the reflection absolutely to zero in terms of film forming performance. Therefore, in order to avoid the excitation of the light source 101 by the return light from the external light guide 150, any one of the following means (1) to (3) is carried out or two or more of the means (1) to (3) are carried out in combination. These three means may be used together with means for adjusting the reflection characteristics of the bandpass filter configuring the multiplexer 113 explained above.
(1) Defocus of the position of the input end of the external light guide 150
(2) Defocus of the position of the light emitting surface of the light source 101
(3) Polarization of emitted light by the light sources 100₁ to 100₃ in one direction

In the second embodiment, at least one of (1) defocus of the position of the input end of the external light guide 150 (a first example) and (2) defocus of the position of the light emitting surface of the light source 101 (a second example) is adopted.

### 5-1. First Example of the Second Embodiment

First, a first example of the second embodiment is explained with reference to FIG. 9 referred to above. A section (b) in FIG. 9 is a schematic diagram illustrating a configuration of an example of a light source device 10b according to the first example of the second embodiment. As illustrated in this figure, in the light source device 10b, the position of the incident end of the external light guide 150 is separated from the lens 122 by an offset Δd₁ with respect to the configuration of the light source device 10a illustrated in the section (a) of FIG. 9 described above.

In the configuration of the light source device according to the embodiment, the lens magnification of the multiplexing system relating to the multiplexer 113 is set such that an image of the emission end of the internal light guide 130 is formed on the incident surface of the external light guide 150 and an image of the light source 101 is also formed on the incident surface of the external light guide 150. For that reason, considering the return light of the image by the internal light guide, the image of the internal light guide 130 is formed on the surface (the incident surface) of the incident end of the external light guide 150 and is formed on the light emitting surface of the light source 101 by two times of the reflection in the bandpass filter in the multiplexer 113.

In general, when a phosphor excitation phenomenon is considered, light density of the excitation light is a factor of emission intensity. Therefore, even if the image by the internal light guide 130 is formed on the light emitting surface of the light source 101, if optical component disposition is finely corrected such that a degree of the image formation decreases, the light density of the excitation light with respect to the light source 101 is reduced.

For example, when the lens 123, which is the final lens of the optical system, and the incident end of the external light guide 150 are separated by the offset Δd₁ within a range having less influence on optical efficiency, the optical path length of the excitation light reaching the light source 101 increases by the offset Δd₁×2. Therefore, a defocus effect increases accordingly.

As explained above, in the first example of the second embodiment, since the offset Δd₁ is given to the position of the incident end of the external light guide 150 to defocus the position, excitation of the light source 101 by the return light of the external light guide 150 can be suppressed.

Note that, in the first example of the second embodiment, a back focus f_{2b} of the lens 122 and a distance Q from the lens 122 to the incident surface of the external light guide 150 preferably satisfy a relation of the following Expression (6).

$${f}_{2 b} \times 0 \text{.9} < Q < {f}_{2 b} \times 1.1 \qquad ( 6 )$$

### 5-2. Second Example of the Second Embodiment

Subsequently, a second example of the second embodiment is explained. FIG. 11 is a schematic diagram illustrating a configuration of an example of a light source device according to the second example of the second embodiment. As illustrated in FIG. 11, in a light source device 10c, the position of the light emitting surface of the light source 101 is separated from the lens 123 by an offset Δd₂ with respect to the configuration of the light source device 10a illustrated in the section (a) of FIG. 9 explained above.

As explained above, the defocus effect can also be obtained by a method of giving the offset Δd₂ to the position of the light source 101. In the second example, optical efficiency at the time when the ray emitted from the light source 101 is taken into the external light guide 150 is deteriorated. For that reason, by giving the offset Δd₂ while keeping the balance of the defocus effect and the optical efficiency, the light density of the excitation light with which the light source 101 is irradiated can be reduced.

Note that, in the second example of the second embodiment, a back focus f_{3b} (see FIG. 9) of the lens 123 and the distance R from the light emitting surface of the light source 101 to the lens 123 preferably satisfy a relation of the following Expression (7).

$${f}_{3 b} \times 0.9 < R < {f}_{3 b} \times 1.1 \qquad ( 7 )$$

As explained above, in the second example of the second embodiment, since the offset Δd₂ is given to the position of the light emitting surface of the light source 101 to defocus the position, the excitation of the light source 101 by the return light of the external light guide 150 can be suppressed.

### 6. Third Embodiment of the Present Disclosure

Subsequently, a third embodiment of the present disclosure is explained. The third embodiment is an example in which (3) the polarization of the light emitted from the light sources 100₁ to 100₃ in one direction explained above is performed.

For example, referring to a section (a) of FIG. 9, it is assumed that a ray in a wavelength band of light B is emitted from the light source 100₁, a ray in a wavelength band of light C is emitted from the light source 100₂, and a ray in a wavelength band of light D is emitted from the light source 100₃. In this case, the reflection characteristic of the bandpass filter in the multiplexer 113 is as illustrated in FIG. 10 referred to above.

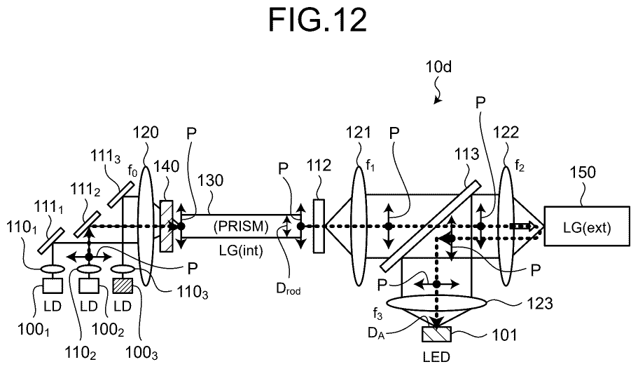

In general, in the principle of a bandpass filter, polarized light, reflection of which is easily suppressed when passing through the bandpass filter, is present. FIG. 12 is a schematic diagram illustrating a configuration of an example of a light source device according to a third embodiment. FIG. 12 illustrates a state in which, in a light source device 10d corresponding to the light source device 10a illustrated in the section (a) of FIG. 9, rays emitted from the light sources 100₁ to 100₃ reach the external light guide 150 and return light from the external light guide 150 reaches the light source 101.

For example, in an optical configuration of the light source device 10d illustrated in FIG. 12, polarized light, reflection of which is easily suppressed by the bandpass filter in the multiplexer 113, is, in general, P-polarized light, a polarization direction of which is the paper surface direction (⇔), with respect to the return light from the external light guide 150. In this case, optical paths are traced back to the light sources 100₁ to 100₃ and polarization directions are aligned such that rays are emitted from the light sources 100₁ to 100₃ in the polarization direction in the paper surface direction. Accordingly, it is possible to create an optical configuration in which the return light by the external light guide 150, with which the light source 101 is irradiated, is suppressed.

When the optical configuration explained above is created, more practically, an S-polarized component is mixed with a P-polarized component in the return light from the external light guide 150. At this time, since polarized light at positions on an optical path is aligned with P-polarized light, the P-polarized light is a main component in the return light as well. Therefore, in the light source device 10d, it is possible to effectively suppress the return light from the external light guide 150 to the light source 101 by considering design priority, for example, performing reflection design with a main constituent being placed on the P-polarized light and controlling the S-polarized component according to necessity.

Note that the effects described in this specification are only illustrations and are not limited. Other effects may be present.

Note that the present technology can also take the following configurations.
(1) A light source device comprising:
   an incident lens on which first light emitted from a first light source is made incident;
   a first light guide on which the first light emitted from the incident lens is made incident;
   a multiplexing unit that multiplexes the first light emitted from the first light guide and second light emitted from a second light source and makes multiplexed light incident on a second light guide; and
   a conversion element that diffuses incident light at a predetermined diffusion angle, wherein
   the conversion element
   is provided between the incident lens and the first light guide.
(2) The light source device according to the above (1), further comprising:
   a first lens, which has a first focal length, for making light emitted from the first light guide incident on the multiplexing unit; and
   a second lens, which has a second focal length, for making light emitted from the second light source incident on the multiplexing unit, wherein
   a ratio of a sectional size of the first light guide and the first focal length and a ratio of a size of a light emitting surface of the second light source and the second focal length are substantially equal.
(3) The light source device according to the above (1) or (2), wherein
   at least one of a sectional size of the first light guide and a size of a light emitting surface of the second light source is equal to or smaller than a size of an incident end of the second light guide.
(4) The light source device according to any one of the above (1) to (3), wherein
   the first light is narrowband light and the second light is wideband light.
(5) The light source device according to any one of the above (1) to (4), wherein
   the predetermined diffusion angle is an angle at which light emitted from the conversion element is diffused at an incident end of the first light guide without exceeding a sectional size of the first light guide.
(6) The light source device according to any one of the above (1) to (5), further comprising
   a third lens, which has a third focal length, for making multiplexed light obtained by multiplexing the first light and the second light in the multiplexing unit incident on the second light guide, wherein
   a distance from the third lens to the second light guide is a distance obtained by giving an offset to the third focal length.
(7) The light source device according to any one of the above (1) to (6), further comprising
   a second lens, which has a second focal length, for making light emitted from the second light source incident on the multiplexing unit, wherein
   a distance from the second lens to the second light source is a distance obtained by giving an offset to the second focal length of the second lens.
(8) The light source device according to any one of the above (1) to (7), wherein
   the light source device is configured to align at least a polarization direction of the first light emitted from the first light source with a polarization direction in the multiplexing unit.
(9) The light source device according to any one of the above (1) to (8), wherein
   the conversion element is any one of a diffusion plate, a fly-eye lens, and a micro-lens array.
(10) The light source device according to any one of the above (1) to (9), wherein
   the first light source is a laser diode and the second light source is a light emitting diode (LED).
(11) An endoscope system comprising:
   an incident lens on which first light emitted from a first light source is made incident;
   a first light guide on which the first light emitted from the incident lens is made incident;
   a multiplexing unit that multiplexes the first light emitted from the first light guide and second light emitted from a second light source and makes multiplexed light incident on a second light guide; and
   a conversion element that diffuses incident light at a predetermined diffusion angle, wherein
   the conversion element includes:
   a light source device provided between the incident lens and the first light guide;
   an imaging device configured to image an imaging range corresponding to an irradiation range irradiated with light emitted from the second light guide; and
   a display device that displays a captured image captured by the imaging device.

### REFERENCE SIGNS LIST

10, 10a, 10b, 10c, 10d, 1000, 5043 LIGHT SOURCE DEVICE
100, 100₁, 100₂, 100₃, 101 LIGHT SOURCE
110, 110₁, 110₂, 110₃ COLLIMATE LENS
111, 111₁, 111₂, 111₃ TOTAL REFLECTION MIRROR
112 DIFFUSION PLATE
113 MULTIPLEXER
120, 121, 122, 123 LENS
130 INTERNAL LIGHT GUIDE
140 PRE-DIFFUSION PLATE
150 EXTERNAL LIGHT GUIDE
200, 210 LIGHT AMOUNT DISTRIBUTION

1. A light source device comprising:
an incident lens on which first light emitted from a first light source is made incident;
a first light guide on which the first light emitted from the incident lens is made incident;
a multiplexing unit that multiplexes the first light emitted from the first light guide and second light emitted from a second light source and makes multiplexed light incident on a second light guide; and
a conversion element that diffuses incident light at a predetermined diffusion angle, wherein
the conversion element
is provided between the incident lens and the first light guide.

2. The light source device according to claim 1, further comprising:
a first lens, which has a first focal length, for making light emitted from the first light guide incident on the multiplexing unit; and
a second lens, which has a second focal length, for making light emitted from the second light source incident on the multiplexing unit, wherein
a ratio of a sectional size of the first light guide and the first focal length and a ratio of a size of a light emitting surface of the second light source and the second focal length are substantially equal.

3. The light source device according to claim 1, wherein
at least one of a sectional size of the first light guide and a size of a light emitting surface of the second light source is equal to or smaller than a size of an incident end of the second light guide.

4. The light source device according to claim 1, wherein
the first light is narrowband light and the second light is wideband light.

5. The light source device according to claim 1, wherein
the predetermined diffusion angle is an angle at which light emitted from the conversion element is diffused at an incident end of the first light guide without exceeding a sectional size of the first light guide.

6. The light source device according to claim 1, further comprising
a third lens, which has a third focal length, for making multiplexed light obtained by multiplexing the first light and the second light in the multiplexing unit incident on the second light guide, wherein
a distance from the third lens to the second light guide is a distance obtained by giving an offset to the third focal length.

7. The light source device according to claim 1, further comprising
a second lens, which has a second focal length, for making light emitted from the second light source incident on the multiplexing unit, wherein
a distance from the second lens to the second light source is a distance obtained by giving an offset to the second focal length of the second lens.

8. The light source device according to claim 1, wherein
the light source device is configured to align at least a polarization direction of the first light emitted from the first light source with a polarization direction in the multiplexing unit.

9. The light source device according to claim 1, wherein
the conversion element is any one of a diffusion plate, a fly-eye lens, and a micro-lens array.

10. The light source device according to claim 1, wherein
the first light source is a laser diode and the second light source is a light emitting diode (LED).

11. An endoscope system comprising:
an incident lens on which first light emitted from a first light source is made incident;
a first light guide on which the first light emitted from the incident lens is made incident;
a multiplexing unit that multiplexes the first light emitted from the first light guide and second light emitted from a second light source and makes multiplexed light incident on a second light guide; and
a conversion element that diffuses incident light at a predetermined diffusion angle, wherein
the conversion element includes:
a light source device provided between the incident lens and the first light guide;
an imaging device configured to image an imaging range corresponding to an irradiation range irradiated with light emitted from the second light guide; and
a display device that displays a captured image captured by the imaging device.
